(19) United States
(12) Patent Application Publication
(10) Pub. No.: US 2022/0305218 A1
(43) Pub. Date: Sep. 29, 2022

(54) LUMINAL OXYGEN DELIVERY AND DEVICE FOR TREATING DISEASES AND CONDITIONS ASSOCIATED WITH STRICT ANAEROBIC MICROORGANISMS

(71) Applicant: Jonathan von Reusner, Philadelphia, PA (US)

(72) Inventor: Jonathan von Reusner, Philadelphia, PA (US)

(21) Appl. No.: 17/701,835

(22) Filed: Mar. 23, 2022

Related U.S. Application Data
(60) Provisional application No. 63/164,714, filed on Mar. 23, 2021.

(51) Int. Cl. A61M 13/00

(52) U.S. Cl. CPC A61M 13/003; A61M 2210/1064; A61M 2202/0208; A61M 2205/3344; A61M 2205/32

(57) ABSTRACT

This disclosure relates to methods and apparatus for treating diseases and conditions of the colon and alimentary canal which are associated with obligate anaerobic microorganisms, and particularly, for treating Clostridioides difficile infection. The method and apparatus provide for continuous administration of intraluminal oxygen to the rectum, colon, and cecum to maintain oxygen in those environments at levels toxic to obligate anaerobic microorganisms such as Clostridioides difficile.

### CROSS REFERENCE TO RELATED APPLICATION

This application claims the benefit of provisional application U.S. Ser. No. 63/164,714, filed on Mar. 23, 2021, which is incorporated herein by reference in its entirety.

### FIELD OF THE DISCLOSURE

This disclosure relates to methods and apparatus for treating diseases and conditions of the colon and alimentary canal which are associated with obligate (strict) anaerobic microorganisms, and particularly, for treating Clostridioides difficile infection. The method and apparatus provide for continuous administration of intraluminal oxygen to the rectum, colon, and cecum to maintain oxygen in those environments at levels which will inhibit growth of obligate anaerobic microorganisms such as Clostridioides difficile.

### BACKGROUND

Clostridioides difficile (formerly Clostridium difficile and commonly referred to as C. difficile or C. diff) is a gram positive, spore forming obligate anaerobic bacterium responsible for significant morbidity and mortality in humans. This organism is found in the human colon as a component of normal flora where oxygen tension is deeply hypoxic, i.e., less than 1 mm Hg. In dysbiosis of the microbiome, C. difficile can cause life threatening diarrhea and colitis via proliferation throughout the colonic mucosa. The most significant risk factors for C. difficile infection (CDI) include antibiotic exposure, particularly broad-spectrum antibiotics including ampicillin, amoxicillin, cephalosporins, clindamycin, and fluoroquinolones (Leffler 2015). Other risk factors include advanced age, gastrointestinal surgery, inflammatory bowel disease, and immunosuppression (Leffler 2015).

Between 1993 and 2016, the incidence of CDI increased 260%, from 25 to 67 cases per 100,000 persons. This increase emerged as a consequence of particularly virulent ribotypes (Elixhauser 2006; CDC Report 2016) and as an artifact of switching from toxin enzyme immunoassays to more sensitive nucleotide amplification testing (NAAT) (Gould 2013). Despite recent reductions in the incidence of CDI, particularly as a result of reduced quinolone use in the hospital setting, CDI remains a major source of morbidity, mortality, and a leading cause of health care associated infections (Guh 2020). While hospital-acquired CDI has declined, community-acquired CDI is increasing and accounted for over 35% of reported cases (Guh 2020). Rises in community-acquired cases may be related to the rise in outpatient medicine (Guh 2020) and the emergence of CDI in animal agriculture (Weese 2006; Songer 2005; Weese 2009). The estimated overall global incidence of CDI in 2019 was 2.24 per 1,000 admissions per year with the US alone reporting 400,000 cases per year and 29,000 deaths at an estimated annual cost of $5.4-6.3 billion (Balsells 2019; Guh 2020).

Adding to this burden is the high rate of disease relapse and antibiotic resistance. While 90% of patients respond initially to antibiotic therapy, the rate of recurrence within 8 weeks of the resolution of symptoms ranges from 20% after an initial episode to 60% after multiple prior recurrences (Fekety 1997; McFarland 1999). The reasons for disease recurrence are not fully understood, but may result from latent C. difficile spores that persist after treatment to then “bloom” under favorable physiological, immunological, and microecological conditions.

Antibiotic-resistant C. difficile strains remain a major public health concern, and further antibiotic exposure drives resistance. The mainstays of CDI treatment, metronidazole and vancomycin, have about a 1% resistance rate (Sholeh 2020). Furthermore, resistance to the antibiotics associated with CDI development including fluoroquinolones, clindamycin, rifampin, and the tetracyclines, is increasing (Sholeh 2020; Peng 2017). There are also wide ranges of resistance detection based on geographic location and study. Overall, there has been an increase in both the detection of strains resistant to metronidazole and vancomycin, as well as the rate of treatment failure with these drugs in the past decade (Peng 2017).

Although antibiotic exposure is a primary risk factor for CDI, they remain the foundation of treatment. Treatment recommendations from the Infectious Disease Society of America (IDSA) guidelines and the Journal of the American Medical Association (JAMA) for patients with a high likelihood of unexplained new-onset watery diarrhea, with either a positive NAAT or a stool toxin test for CDI, depend on several factors (McDonald 2018). Moreover, treatments differ for patients experiencing primary CDI (pCDI) or recurrent infection (rCDI), and by whether the patient meets the criteria for fulminant or nonfulminant infection as defined by the clinical signs of hypotension/shock, ileus, and/or megacolon (McDonald 2018).

For fulminant and nonfulminant cases of pCDI, enteral vancomycin is the first recommended treatment in addition to supportive IV fluids, electrolytes, and necessary antipyretics. For nonfulminant pCDI, recommendations also include the use of fidaxomicin, a newer narrow spectrum antibiotic. In cases of fulminant CDI, recommendations include oral or nasogastric tube delivery of high dose vancomycin in addition to intravenous (iv) metronidazole (McDonald 2019; Sailhamer 2009).

Approximately 1% of all CDI cases and 30% of fulminant cases require emergency surgery, which is reserved for patients with severe colitis who fail to improve with medical therapy, who have generalized peritonitis, or who develop colonic perforation in the setting of fulminant CDI or toxic megacolon. The average 30-day postoperative mortality is high, at 41.3% with a range of 19-71% depending on the case and specific procedure conducted (Bhangu 2012).

Treatment for rCDIs, which account for 20-60% of CDI cases, remain a challenge hindered by either weak evidence for effectiveness of antibiotic therapy, or the high mortality and costs associated with surgery. In the absence of data to compare non-antibiotic to antibiotic treatments for pCDI cases, it remains unclear whether antibiotic therapy for pCDI is in itself a driver of rCDI. Given that antibiotic exposure is the primary risk factor for pCDI and that public health efforts to promote more conservative use of antibiotics have proven successful in reducing the overall CDI incidence in recent years, treatment modalities that reduce the reliance on antibiotics for the overall treatment of CDI are worth pursuing.

Fecal microbiota transplant (FMT) is an alternative treatment modality gaining recognition for treating recurrent CDI. In FMT, stool from a healthy donor is infused directly into the colonic lumen of a person infected with CDI to restore the dysbiosis thought to cause CDI. In a landmark study, CDI resolved within 10 weeks for 81% of patients with rCDI that received a dose of vancomycin followed immediately by FMT. While FMT is effective, challenges remain including scalability, quality assurance, donor-recipient matching, as well as the technical aspects of stool preparation, storage, and delivery.

Accordingly, additional methods of treating CDI are needed. As a strict, obligate anaerobe, C. difficile is unable to grow at oxygen partial pressure of ˜3 mm Hg or greater, and can only persist in a vegetative state in room air (20% oxygen, 160 mm Hg) for ˜24 hrs (Fairweather 2016). While this extreme oxygen aversion enables C. difficile and other obligate anaerobes to persist in the deeply hypoxic environment of the colon, where physiological oxygen tension is typically below 1 mm Hg, increased oxygen tension reduces vegetative cell viability, leading to fewer spores and less toxin formation. Moreover, suppressing growth of obligate anaerobic organisms, including C. difficile, with oxygen may not only reduce total spore burden but can allow aerotolerant commensal flora to expand and establish ecological dominance, providing further benefit for the treatment of CDI, reduction of gut-associated inflammation and other associated conditions. The present invention addresses this need by administration of a continual flow of intraluminal oxygen to the rectum, colon, and cecum to maintain oxygen in those environments at levels toxic to C. difficile and other strictly anaerobic microorganisms.

### SUMMARY OF THE DISCLOSURE

The present invention relates to a treatment modality and apparatus for the continuous, gentle insufflation of the colon, with low flow concentration of oxygen from about 21 to about 100% and providing continuous pressure release and equalization to maintain an oxygenated colonic environment. The method and apparatus are useful for treating diseases and conditions of the colon and alimentary canal which are associated with obligate anaerobic microorganisms, including CDI, irritable bowel syndrome, dysbiosis of the colon, inflammatory conditions, and the like.

In one aspect, the invention is directed to a method of inhibiting growth of obligate anaerobic microorganisms present in the lumen of the colon of a subject which comprises (a) administering a gas comprising from about 21% to about 100% oxygen via the anus into the lumen of the colon at a gentle/low flow rate to establish an oxygenated colonic environment, (b) providing for continuous gas release via the anus to provide substantially equalized internal and external pressure, and (c) maintaining this environment for a time sufficient to inhibit growth of obligate anaerobic microorganisms. By inhibiting growth of obligate anaerobes, the number and/or diversity of beneficial microorganisms may be increased to allow establishment or reestablishment of healthy gut flora. The method is particularly useful for inhibiting the vegetative growth phase of C. difficile and lead to reduced or limited spore formation.

Another aspect of the invention provides a method of treating a disease or disorder of the colon associated with obligate anaerobic microorganisms in a subject which comprises (a) administering a gas comprising from about 21% to about 100% oxygen via the anus into the lumen of the colon at a flow rate to establish an oxygenated colonic environment, (b) providing for continuous gas release via the anus to equalize internal and external pressure, and (c) maintaining that oxygenated environment for a time sufficient to treat the disease or disorder. Without being bound by a mechanism, inhibiting growth of the obligate anaerobes may allow proliferation and increased diversity of beneficial microorganisms in the colon, allowing those microorganisms to out compete the anaerobes and to reestablish a healthy gut flora. The method is used to treat one or more C. difficile infection (CDI), irritable bowel syndrome, diseases related to or arising from dysbiosis of the colon, their associated inflammatory conditions, and the like.

In some embodiments, the flow rate of the gas ranges from about 0.1 L/min to about 5 L/min. In some embodiments, the flow rate of the gas produces a partial pressure of oxygen of at least 5 mm Hg in the colon. An oxygen partial pressure of 5 mm Hg or greater in the colon is sufficient to establish an oxygenated colonic environment. In some embodiments, gas is insufflated for about 30 minutes, about 1 hour, about 2 hours, about 4 hours, about 8 hours, about 16 hours, about 1 day, about 2 days, about 3 days, about 4 days, about 5 days, about 6 days, to about a week, or about 10 days. In preferred embodiments, the insufflation time ranges from about 30 minutes to about 2 hours or from about 1 hour to about 4 hours. In other embodiments, insufflating is repeated from 2 to four times per day for about 30 minutes to about 4 hours per treatment.

A further aspect of the invention provides a method of treating a disease or disorder of the colon associated with obligate anaerobic microorganisms in a subject which comprises (a) positioning an insufflation device comprising an anal stopper fitted with an outflow tube and an oxygen inlet tube, through the anus and into the colon of the subject by securing the stopper at the anal canal opening and advancing the inlet tube into the colon to a desired location between the iliocecal valve and the anus; (b) gently insufflating a gas comprising from about 21% to 100% oxygen into the colon through the inlet tube at a flow rate sufficient to increase the oxygen partial pressure in the colon while providing continuous gas release via the outflow tube; and (c) maintaining the gas flow and/or pressure for sufficient time to inhibit growth of the anaerobic microorganisms. Again, without being bound by a mechanism, inhibiting growth of the obligate anaerobes may allow proliferation and increased diversity of beneficial microorganisms in the colon, allowing those microorganisms to out compete the anaerobes and to reestablish a healthy gut flora. The method is used to treat one or more C. difficile infection (CDI), irritable bowel syndrome, diseases related to or arising from dysbiosis of the colon, their associated inflammatory conditions, and the like.

In an embodiment, use of the apparatus in the method provides a flow rate of the gas from about 0.1 L/min to about 5 L/min. In some embodiments, the flow rate of the gas produces a partial pressure of oxygen of at least 5 mm Hg in the colon. In some embodiments the partial pressure of oxygen ranges from about 5 mm Hg to about 10 mm Hg or greater. In some embodiments, gas is insufflated for about 30 minutes, about 1 hour, about 2 hours, about 4 hours, about 8 hours, about 16 hours, about 1 day, about 2 days, about 3 days, about 4 days, about 5 days, about 6 days, to about a week, or about 10 days. In preferred embodiments, the insufflation time ranges from about 30 minutes to about 2 hours or from about 1 hour to about 4 hours. In other embodiments, insufflating is repeated from 2 to four times per day for about 30 minutes to about 4 hours per treatment.

According to one aspect of the invention there is provided an insufflation device for introducing oxygen into the colon of an individual suffering from a disease or disorder that would benefit from a more highly oxygenated environment in the colon, or any part thereof, for time sufficient to treat said disease or disorder, e.g., a disease or disorder associated with obligate anaerobic microorganisms. In one embodiment the insufflation device comprises an anal stopper adapted to form a gas-tight seal with an anus of an organism, wherein the anal stopper comprises an inlet port and an outlet port; and a gas delivery tube comprising a distal portion adapted to be positioned within a lumen of a colon of the organism, wherein the gas delivery tube extends through the inlet port of the stopper and is adapted to be positioned along at least a portion of a colon of the organism.

In further embodiments, the insufflation device further comprises a source of a gas, wherein the proximal end of the gas delivery tube is connected an output of the source and wherein the gas delivery tube comprises an inner lumen and one or more orifices extending from the inner lumen to a space surrounding the gas delivery tube, wherein, when the gas delivery tube extends along at least a portion of the colon of the organism, the gas flows from the source through the inner lumen and out from the inner lumen through the one or more orifices, wherein the gas delivery tube is slidingly engaged with the inlet port and movable in a proximal and a distal direction to adjust a length of a distal portion of the gas delivery tube along the colon.

In accordance with some embodiments, the stopper comprises an outflow tube extending through outlet port, wherein, when the stopper is disposed in the anus of the organism and the distal portion of the gas delivery tube is disposed in the colon, the gas flows outward from the colon through the outflow tube. In other embodiments, the stopper comprises an outflow tube extending through outlet port, wherein, when the stopper is disposed in the anus of the organism and the distal portion of the gas delivery tube is disposed in the colon, the gas flows through the one or more orifices into the colon of the organism and outward from the colon through the outflow tube.

In some embodiments, the gas source comprises a pressure converter, wherein the pressure converter receives the gas at the first pressure and outputs the gas from the source at a second pressure.

In other embodiments, the outflow tube comprises a pressure regulator, wherein the pressure regulator maintains a pressure of the gas in the colon at a treatment pressure.

In further embodiments, the stopper comprises an inflatable portion adapted to receive an inflation fluid or gas, wherein, when the stopper is positioned in the anus and when inflation fluid or gas is forced into the inflatable portion, a surface of the stopper is pressed against a wall of the anus to form a gas-tight seal between the surface of the stopper. and the anus.

Yet other embodiments provide that the outflow tube of the disclosed device further comprises, is attached or is adapted to be fed into a collection container, wherein solid or liquid substances flowing in a proximal direction through the outflow tube are collected in the collection container.

According to another aspect, the one or more orifices of the inlet tube comprise a plurality of sets of orifices, wherein a first set is positioned a first distance along the length of the tube from the stopper, wherein the first distance corresponds with a distance along the colon from the anus to a descending colon of the organism. In other embodiments, the one or more orifices further comprise a second set of orifices is positioned a second distance along the length of the tube from the stopper, wherein the second distance corresponds with a distance along the colon from the anus to a transverse colon of the organism. In some embodiments, the one or more orifices comprise an opening at a distal end of the inlet tube. In some embodiments, the inlet tube comprises any combination of the one or more orifices

According to a further aspect, the device includes a flow meter that provides an indication of the flow rate of the gas into and out from the colon, that is the flow meter can measure the flow rate of the gas through the inner lumen.

In some embodiments, the gas delivery tube further comprises one or more radio-opaque markers.

In some embodiments, the devices of the invention further comprise a safety valve connected with the gas delivery tube to monitor the gas pressure in the inner lumen and that opens to vent the inner lumen of excess gas if the pressure exceeds a selected pressure. In some embodiments of the device, the gas source comprises a gas mixing valve.

In an embodiment the device for treating a disease or disorder of the colon associated with obligate anaerobic microorganisms comprises:

a source of a gas at a first pressure;

a pressure converter connected with the source, wherein the pressure converter receives the gas at the first pressure and outputs the gas at a second pressure;

a gas delivery tube, wherein a proximal end of the gas delivery tube is connected an output of the pressure converter and wherein the gas delivery tube comprises an inner lumen and one or more orifices extending from the inner lumen to a space surrounding the gas delivery tube; and

a stopper adapted to engage with and form a gas-tight seal with an anus of the organism, wherein the stopper comprises an inlet port and an outlet port,

wherein the gas delivery tube extends through the inlet port and is adapted to be positioned along at least a portion of the colon of the organism, wherein the gas flows from the source, through the converter, through the inner lumen and out from the inner lumen through the one or more orifices, wherein the gas delivery tube is slidingly engaged with the inlet port and movable in a proximal and a distal direction to adjust a length of a distal portion of the gas delivery tube extending from the stopper.

### BRIEF DESCRIPTION OF THE DRAWINGS

FIG. 1 shows an insufflation device according to an embodiment of the disclosure.

FIG. 2 shows a side view of an anal stopper of the embodiment of FIG. 1.

FIG. 3 is a cross section view of an anal stopper of the embodiment of FIG. 1.

### DETAILED DESCRIPTION OF THE INVENTION

### Definitions

In order that the present invention may be more readily understood, certain terms are defined below. Additional definitions may be found within the detailed description of the invention.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

The singular forms “a,” “an,” and “the” include the plurals unless the context clearly dictates otherwise.

The term “including” is used to mean “including but not limited to.” “Including” and “including but not limited to” are used interchangeably.

The terms “patient,” “subject,” and “individual” may be used interchangeably and refer to either a human or a non-human animal. These terms include mammals such as humans, primates, livestock animals (e.g., bovines, porcines), companion animals (e.g., canines, felines) and rodents (e.g., mice and rats). Typically, the terms “subject” and “patient” are used interchangeably herein in reference to a human subject.

The term “non-human mammal” means a mammal which is not a human and includes, but is not limited to, a mouse, rat, rabbit, pig, cow, sheep, goat, dog, primate, or other non-human mammals typically used in research.

For purposes of this disclosure, the terms “distal,” “distally,” “distal of” and the like will be used throughout this disclosure to refer to the direction or relative position away from the operator of the device and toward the body of a patient being treated using the device. The terms “proximal,” “proximally,” “proximal of” and the like will be used throughout this disclosure to refer to the direction toward the operator of the device and away from the body of a patient being treated using the device.

As used herein, “treating” or “treatment” and grammatical variants thereof refer to an approach for obtaining beneficial or desired clinical results. The term may refer to slowing the onset or rate of development of a condition, disorder or disease, reducing or alleviating symptoms associated with it, generating a complete or partial regression of the condition, or some combination of any of the above. For the purposes of this invention, beneficial or desired clinical results include, but are not limited to, reduction or alleviation of symptoms, diminishment of extent of disease, stabilization (i.e., not worsening) of state of disease, delay or slowing of disease progression, amelioration or palliation of the disease state, and remission (whether partial or total), whether detectable or undetectable. “Treatment” can also mean prolonging survival relative to expected survival time if not receiving treatment. A subject (e.g., a human) in need of treatment may thus be a subject already afflicted with the disease or disorder in question. The term “treatment” includes inhibition or reduction of an increase in severity of a pathological state or symptoms relative to the absence of treatment and is not necessarily meant to imply complete cessation of the relevant disease, disorder or condition.

As used herein, the terms “preventing” and grammatical variants thereof refer to an approach for preventing the development of, or altering the pathology of, a condition, disease or disorder. Accordingly, “prevention” may refer to prophylactic or preventive measures. For the purposes of this invention, beneficial or desired clinical results include, but are not limited to, prevention or slowing of symptoms, progression or development of a disease, whether detectable or undetectable. A subject (e.g., a human) in need of prevention may thus be a subject not yet afflicted with the disease or disorder in question. The term “prevention” includes slowing the onset of disease relative to the absence of treatment and is not necessarily meant to imply permanent prevention of the relevant disease, disorder or condition. Thus “preventing” or “prevention” of a condition may in certain contexts refer to reducing the risk of developing the condition or preventing or delaying the development of symptoms associated with the condition.

As used herein, an “effective amount,” “therapeutically-effective amount” or “effective dose” is an amount of a composition (e.g., a therapeutic composition or agent) that produces at least one desired therapeutic effect in a subject, such as preventing or treating a target condition or beneficially alleviating a symptom associated with the condition.

The most desirable therapeutically effective amount is an amount that will produce a desired efficacy of a particular treatment selected by one of skill in the art for a given subject in need thereof. This amount will vary depending upon a variety of factors understood by the skilled worker, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, namely by monitoring a subject's response to administration of a compound and adjusting the dosage accordingly. See, e.g., Remington: The Science and Practice of Pharmacy 21st Ed., Univ. of Sciences in Philadelphia (USIP), Lippincott Williams & Wilkins, Philadelphia, Pa., 2005.

All numbers in this disclosure indicating amounts, ratios of materials, physical properties of materials, and/or use are to be understood as modified by the word “about,” except as otherwise explicitly indicated. The term “about” when referring to a number or a numerical range means that the number or numerical range referred to is an approximation within experimental variability (or within statistical experimental error), and thus the number or numerical range can vary from, for example, between 1% and 15% of the stated number or numerical range.

Insufflation is the act of blowing something (such as a gas, powder, or vapor) into a body cavity. Insufflation has many medical uses, most notably as a route of administration for various drugs.

As used herein, an oxygenated environment is established in the colon, rectum, or any section(s) thereof, when the oxygen level increases sufficiently to retard, restrict, or inhibit the growth of obligate anaerobes. Under normal physiological conditions, i.e., in the absence of treatment or prior to treatment, oxygen partial pressure in the rectum or colon is typically 1-5 mm and can range upward to about 10 mm or more. In accordance with the present invention, administering air or an oxygen-containing gas increases the oxygen concentration above at least about 8-10%, at which point growth inhibition of obligate anaerobes is evident. For example, an oxygen partial pressure of 5 mm Hg or greater in the colon is sufficient to establish an oxygenated colonic environment.

### Methods

The present invention relates to a treatment modality involving the gentle insufflation of the cecum, colon and rectum of humans or other mammalian species, by administering oxygen (21-100%) at a low flow rate with continuous pressure release and equalization to the external world, with or without additional gases admixed over a period of 30 minutes to 10 days to treat CDI or other diseases and conditions of the colon and alimentary canal which are associated with strict (obligate) anaerobic microorganisms. The oxygen may be delivered as air (at ˜21% oxygen) or as supplemental oxygen. Supplemental oxygen can be admixed with air, hydrogen, nitrogen, helium, carbon dioxide, humidified water, or other medically-acceptable or therapeutic gases that are useful to assure the desired oxygen concentration or beneficial in the treatment of CDI. This treatment may be employed to treat primary CDI, recurrent CDI, fulminant CDI, and/or non-fulminant CDI as defined in humans and veterinary medicine. This therapy may be employed as monotherapy, or as adjuvant therapy before, during, or after other treatment modalities including but not limited to antibiotic regimens, FMT, and/or hyperbaric therapy. The methods of the invention may also be used as adjuvant therapy before and/or after surgical intervention. This treatment may be administered once, or multiple times as needed. This treatment may also be employed in the veterinary setting. This technique may be applied to other inflammatory bowel diseases that benefit from elevated levels of luminal oxygen.

Accordingly, one method of the invention is directed to inhibiting growth of obligate anaerobic microorganisms in the colon of a patient which comprises (a) administering a gas, including air, comprising from about 21% to about 100% oxygen via the anus into the lumen of the colon at a gentle/low flow rate to establish an oxygenated environment, (b) providing for continuous gas release via the anus to equalize internal and external pressure, and (c) maintaining the hypoxic environment for a time sufficient to inhibit growth of said microorganisms. By inhibiting growth of the microorganisms, the method can increase the number and/or diversity of beneficial microorganisms in the colon. In some embodiments, the method is used to inhibit vegetative growth of C. difficile which in turn should reduce the spore burden.

In accordance with the method, the flow rate ranges from about 0.1 L/min to about 5 L/min. A partial pressure of oxygen of at least 5 mm Hg in the colon is sufficient to inhibit growth of obligate anaerobes. As used herein the terms, strict anaerobes and obligate anaerobes are interchangeable.

The air or the oxygen-containing gas is administered to the subject to establish the oxygenated colonic for a time of about 30 minutes, about 1 hour, about 2 hours, about 4 hours, about 8 hours, about 16 hours, about 1 day, about 2 days, about 3 days, about 4 days, about 5 days, about 6 days, to about a week, or about 10 days. The treatments of shorter time duration, e.g., 30 min to a few hours, can be repeated multiple times per day, typically from 2-6 times per day.

Another aspect of the invention is directed to methods of treating a disease or disorder of the colon associated with obligate anaerobic microorganisms in a subject which comprises (a) administering a gas comprising from about 21% to about 100% oxygen via the anus into the lumen of the colon at a flow rate to establish an oxygenated colonic environment, (b) providing for continuous gas release via the anus to equalize internal and external pressure, and (c) maintaining the environment for a time sufficient to treat said disease or disorder.

Without being bound by a mechanism, inhibiting growth of the obligate anaerobes will permit proliferation and increased diversity of beneficial microorganisms in the colon, allowing those microorganisms to out compete the anaerobes and to reestablish a healthy gut flora. The method is used to treat one or more C. difficile infection (CDI), irritable bowel syndrome, dysbiosis of the gut, inflammatory conditions and the like. In some embodiments the CDI includes, but is not limited to, primary CDI, recurrent CDI, fulminant CDI, and/or non-fulminant CDI in humans or mammals.

In some embodiments, the flow rate of the gas ranges from about 0.1 L/min to about 5 L/min. In some embodiments, the flow rate of the gas produces a partial pressure of oxygen of at least 5 mm Hg in the colon. In some embodiments, gas is insufflated for about 30 minutes, about 1 hour, about 2 hours, about 4 hours, about 8 hours, about 16 hours, about 1 day, about 2 days, about 3 days, about 4 days, about 5 days, about 6 days, to about a week, or about 10 days. In preferred embodiments, the insufflation time ranges from about 30 minutes to about 2 hours or from about 1 hour to about 4 hours. In other embodiments, insufflating is repeated from 2 to four times per day for about 30 minutes to about 4 hours per treatment.

In some embodiments, the method of treating a disease or disorder of the colon associated with obligate anaerobic microorganisms in a subject employs an insufflation device of the invention or other device adapted to deliver oxygen in the amounts and with continual pressure equalization as provided herein.

In an embodiment, the method of treating a disease or disorder of the colon associated with obligate anaerobic microorganisms in a subject comprises (a) positioning an insufflation device comprising an anal stopper fitted with an outflow tube and an oxygen inlet tube, through the anus and into the colon of the subject by securing the stopper at the anal canal opening and advancing the inlet tube into the colon to a desired location between the iliocecal valve and the anus. The anal stopper forms a patent seal with the anus so that the gas inflow and outflow occur via the inlet and outlet tubes. Once the device is place, pure medical grade oxygen or any medical grade gas admixture containing of oxygen comprising from about 21% to 100% oxygen is gently insufflated into the lumen of the colon through the inlet tube at a flow rate sufficient to increase the oxygen partial pressure in the colon to a degree capable of inhibiting vegetative growth of obligate anaerobes and to produce a oxygenated environment. Simultaneously, any buildup (or potential buildup) of pressure is relieved by continuous gas release through the outflow tube. Such pressures are maintained for a time sufficient to inhibit vegetative growth of obligate anaerobic microorganisms that may be present in the colon or for a time to aid in amelioration of any inflammatory conditions associated with such microorganisms.

Such methods using a device or apparatus of the invention, is practiced in accordance with the other methods of the invention.

Another aspect of the invention is directed to a method of treating C. difficile infection (CDI) in a subject which comprises insufflating an oxygen-containing gas into the lumen of the colon via rectally administering the gas at a flow rate between about 0.1 L/min and 5 L/min to provide an pressure in the colon of at least about 2 mm Hg and no more than 40 mm Hg. Such treatment establishes a oxygenated environment in the lumen of the colon and provides for continuous gas release via the anus to equalize internal and external pressure. By maintaining this environment for sufficient time sufficient the vegetative growth of obligate anaerobes, such as t C. difficile, can be inhibited. This treatment can use a device or apparatus of the invention as described herein

In accordance with the method, the gas delivered to the colon comprises about 21% to 100% oxygen. Physiologic activity by the tissues of the colon removes free oxygen from the lumen of the colon so that providing a continuous flow of oxygen-containing gas at a selected oxygen concentration to the colon assures that a oxygenated atmosphere is maintained in the colon for a sufficient time to interrupt the life cycle strict anaerobes such as C. difficile.

In any of the foregoing embodiments, the oxygen inlet tube may be inserted to the cecum, however placement of the distal end to more proximal sites at the ascending colon, transverse colon, or descending colon is also contemplated. Placement of the oxygen inlet tube to provide the desired therapeutic benefit can be determined by those of skill in the art using art recognized techniques such as fluoroscopy to ascertain the location of the tube in the colon.

### Insufflation Device

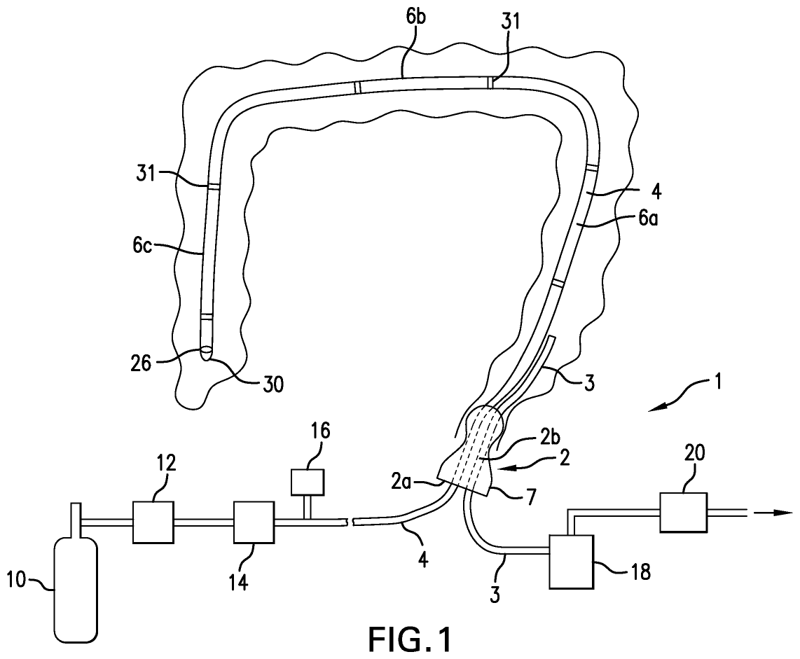

FIG. 1 shows the insufflation device 1 which comprises an anal stopper 2 fitted with an outflow tube 3 and a gas delivery tube 4. A source 10 of a therapeutic gas or gas mixture is provided. The source 10 may be a pressurized gas bottle, an institutional gas system, an air pump, a mechanical oxygen concentrator, or the like. In accordance with the invention, source 10 provides a therapeutic gas mixture for treatment of diseases and conditions of the invention and particularly for CDI. According to some embodiments, the gas comprises between about 21% to about 100% oxygen (O₂). According to some embodiments, the gas may be oxygen admixed with air, hydrogen, nitrogen, helium, carbon dioxide, or other gases. According to a further embodiment, the gas contains water vapor. According to still further embodiments, the gas contains one or more additional therapeutic gases, including but not limited to nitrous oxide, an anesthetic gas or anesthetic gas mixture, to treat C. difficile infection or other diseases or conditions of the rectum, colon, or cecum associated with proliferation of obligate anaerobes, where the increased oxygen tension provides therapeutic benefit in treating those disease and conditions.

A pressure converter 12 is connected with gas source 10. The pressure converter 12 may consist of one or more stages to reduce the gas pressure from the source 10 to a selected pressure for administration to the patient or subject. According to some embodiments, the pressure of the gas is slightly higher than atmospheric pressure to gently inflate the colon. According to one embodiment, converter 12 reduces pressure from the source 10 to a gauge pressure between about 0.1 mm Hg and about 40 mm Hg, with preferred pressures of from about 2 to about 8 or from about 5 to about 10 mm Hg. The gas pressure provided by converter 2 is selected so that a flow of gas is provided through the lumen of the colon. According to one embodiment, the gas pressure does not inflate the colon. According to other embodiments, the pressure inflated the colon slightly to facilitate the flow of gas in and out of colon.

According to one embodiment, output of converter 12 is connected with flow meter 14. Flow meter may comprise a mechanical mechanism to measure gas flow, for example, using a paddle wheel, vane, or turbine or vertically displacing a weighted bobbin, or may comprise a thermal mass, ultrasonic, or vortex-type flow meter. Gas delivery tube 4 is connected with the output of the flow meter 14.

According to other embodiments, instead of having a flow meter 14 interposed between pressure converter 12 and gas delivery tube 4, gas delivery tube 4 is connected directly with the output of converter 12. Flow through tube 4 may be monitored using a non-contact type flow meter through the wall of tube 4, for example, a clip-on ultrasonic-type flow meter.

According to one embodiment, connection between gas delivery tube 4 and pressure converter 12 or flow meter 14 is a removable, medically-suitable connector, for example, a Luer Lock connector or the like.

According to one embodiment, delivery tube 4 includes a safety valve 16. Safety valve 16 remains closed as long as the pressure within tube 4 is less than a safety threshold pressure. If the pressure in tube 4 (and hence within the colon of the individual undergoing treatment) exceeds the safety threshold, valve 16 opens to vent pressure within tube 4. This relieves gas pressure within the colon. According to one embodiment, safety valve 16 prevents pressure in tube 4, and therefore the pressure within the individual's insufflated colon, to exceed about 40 mm Hg above atmospheric pressure.

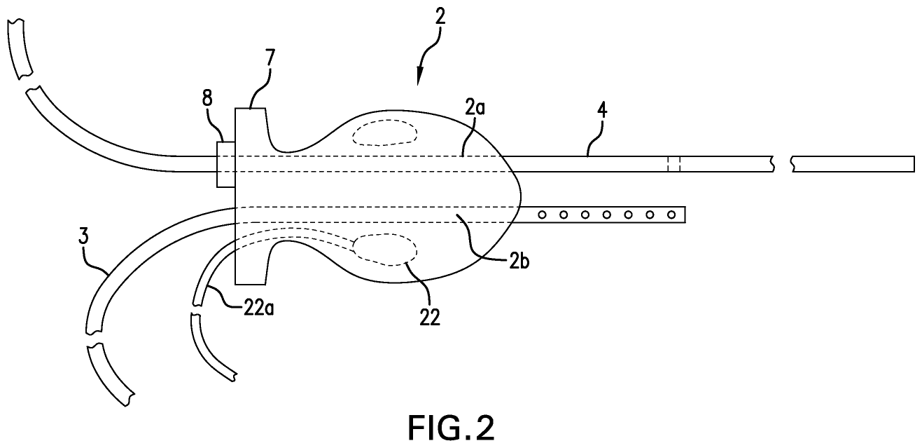

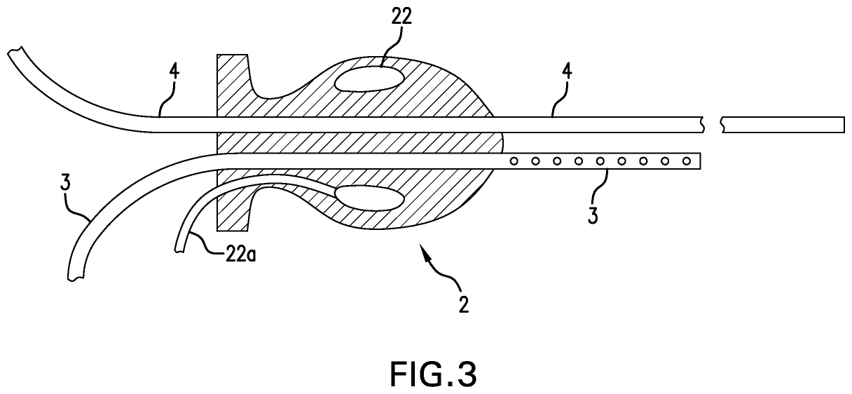

As shown in FIG. 1, stopper 2 is adapted to be fitted into the anus of the patient. FIG. 2 shows a detailed view of stopper 2. FIG. 3 shows a cross section of stopper 2. Stopper 2 is sized to fit into and seal against the anus, rectum, or wall of the colon of the patient. When so fitted, stopper 2 forms a barrier to control the flow of gases into and out from the colon. The stopper 2 may be formed from a medically-suitable material that is biocompatible with the anus of the patient. Stopper 2 may be formed from a medically-acceptable, elastomeric material, for example, silicone, polyurethane, a biocompatible polymer membrane or film, and the like. Such materials can generally conform to ISO10993 and US FDA guidelines for biocompatibility. Other suitable materials, include but are not limited to, polyethylene, polypropylene, polytetrafluoroethylene (PTFE), fluorinated ethylene propylene (FEP), perfluoroalkoxy alkane (PFA), polyvinyl chloride (PVC), polyether ether ketone (PEEK), a liquid crystal polymer, ethylene tetrafluoroethylene (ETFE), a co-polymer or ter-polymer such as tetrafluoroethylene, hexafluoropropylene and vinylidene fluoride ter-polymer (THV), and other polymers such as Tygon®, Pebax®, and combinations thereof.

According to some embodiments, stopper 2 is provided with a lubricious coating to facilitate insertion into the anus. According to some embodiments stopper 2 has a tapered shape, such as the shape of a bottle or test tube stopper.

According to one embodiment, the stopper has a first, unexpanded configuration that has a narrower cross section to facilitate insertion into the anus, and a second, expanded configuration to form a seal once the stopper has been inserted. As shown in FIG. 2 stopper 2 includes inflatable portions 22. An inflation tube 22a is provided to allow pressurized gas or fluid to be force into inflatable portion 22 to expand stopper 2 to facilitate forming a seal with the anus, rectum, or wall of the colon of the patient. Inflatable portion 22 may be an annular cavity within stopper 2. According to one embodiment, the stopper 2 has a lip 7 that resides outside of the body such that it protrudes over the anal opening and helps to hold the stopper at the anal opening and prevents the stopper from moving completely through the anus. Once treatment is completed, the pressurized gas or fluid in cavity 22 is withdrawn via tube 22a, deflating portions 22 to allow the stopper 2 to be removed from the anus.

The stopper 2 has inlet port 2a and outlet port 2b. The gas delivery tube 4 extends through inlet port 2a. According to one embodiment, inlet port 2a is sized to allow tube 4 to be moved proximally and distally through stopper 2 to adjust distance tube 4 extends in the distal direction into the colon. A sealing ring 8 may be provided to form a gas tight seal between stopper 2 and the outer surface of gas delivery tube 4.

Delivery tube 4 is a flexible tube designed to be inserted through the anus and along the colon. By moving tube 4 proximally and distally through inlet port 2a relative to stopper 2, the extent of the tube through the colon can be adjusted. This adjustment can be made before the tube 4 and stopper 2 are inserted through the anus and colon. According to some embodiments the portion of tube 4 extending in the distal direction from stopper 2 is adjusted based on evaluation of the length of the individual's colon using radiographic techniques so that the distal end of tube 4 extends into or near to the ileocecal valve. According to other embodiments, the length of the distal potion of tube 4 extending form stopper 2 is selected based on patient demographic averages.

The length of the distal portion of tube 4 extending from stopper 2 can be adjusted after the tube 4 and stopper 2 have been inserted into the patient's anus and colon. According to one embodiment as shown in FIG. 1, a radio-opaque marker 30 is provided at the end of tube 4 to allow the position of tube 4 in the colon to be visualized using a medical imaging system, for example, using a fluoroscope, so that the end of tube 4 is at or near the ileocecum valve. Other radio-opaque markers 31 may be provided at other portions of tube 4 to aid in locating tube 4 relative to portions of the colon, as will be described below.

According to one embodiment, tube 4 is formed from a medically-suitable material, including medical-grade flexible tubing and the like. An outer diameter of tube 4 is selected to fit within and extend through the colon without causing tissue damage. According to one embodiment, tube 4 has dimensions and mechanical characteristics suitable for insertion through the anus and along the colon. The material and the dimensions of tube 4 are selected so that the tube has sufficient patency that it can be inserted along the colon without kinking and sufficient flexibility to navigate through turns in the colon. According to one embodiment, tube 4 has an inner lumen diameter between about 0.1 mm and 1.0 cm and an outer diameter between about 0.2 mm and 1.2 cm. According to a preferred embodiment, tube 4 has an inner diameter between about 1 mm and about 5 mm and an outer diameter between about 2 mm and about 7 mm. According to a most preferred embodiment, tube 4 has an inner diameter of about 2 mm and an outer diameter of about 5 mm. The outer surface if tube 4 may be provided with a lubricious coating to facilitate passage through the anus and along the colon.

The overall diameter of the stopper 2 is sized to fit securely into the anus of the patient. According to one embodiment, stopper 2 has a maximum diameter between about 1 cm and 15 cm so that a gas-tight seal can be achieved with the anus of the subject. According to a preferred embodiment, stopper 2 has a maximum diameter between about 3 cm and about 7 cm. According to a most preferred embodiment, stopper 2 has a maximum diameter of about 3.5 cm. When stopper 2 is inflatable, its volume can range from about 14 to about 180 ml (assuming an inflatable sphere), and is preferably about 22.5 ml.

The outflow tube 3 extends through outlet port 2b of stopper 2. Outflow tube 3 is made of a flexible, medical or surgical grade tubing or other biocompatible materials such as used for tube 4. According to one embodiment, a portion of the distal end may be made of a more rigid material, and the remaining proximal portion of tube may be made of a more flexible material. According to one embodiment, the length of the distal portion of tube 3 will range from 0.5 cm to 10 cm in length. According to one embodiment, tube 3 has a minimum diameter of 1 mm and a maximum diameter of 5 cm. Tube 3 forms a seal with the inner diameter of outlet port 2b of stopper 2. According to one embodiment, outflow tube 3 extends through the anus and into the anus, rectum or as far as the descending colon of the individual being treated.

The proximal portion of the outflow tube 3 dissipates the gas into the atmosphere. According to one embodiment, the proximal end of outflow tube 3 is connected with a collection container 18. Container 18 may be a solid jar or a collection bag suitable for holding solid or liquid matter expelled from the colon during treatment. The container 18 may be designed to allow solids and liquids to collect in the container and for gases to vent from the container so that pressure within outflow tube 3 is at or near atmospheric pressure.

According to one embodiment, gas delivery tube 4, stopper 2 and outflow tube 3 may be formed from materials that conform to ISO10993 and US FDA guidelines for biocompatibility. According to some embodiments, tubes 3, 4 and stopper 2 are formed from one or more medically-suitable polymers known and available in the art, including but not limited to polyethylene, polypropylene, polytetrafluoroethylene (PTFE), fluorinated ethylene propylene (FEP), perfluoroalkoxy alkane (PFA), polyvinyl chloride (PVC), polyether ether ketone (PEEK), a liquid crystal polymer, ethylene tetrafluoroethylene (ETFE), a co-polymer or ter-polymer such as tetrafluoroethylene, hexafluoropropylene and vinylidene fluoride ter-polymer (THV), and other polymers such as Tygon®, Pebax®, and combinations thereof.

According to one embodiment, collection container 18 is connected with a pressure regulator 20. Pressure regulator 20 restricts the flow of gas from outlet tube 3 to maintain a selected internal pressure within the colon of the patient. According to another embodiment, pressure regulator 20 is adapted to provide a vacuum or a pressure slightly lower than the pressure within tube 4 and within the colon to draw gas from the colon and prevent the accumulation of luminal pressure. According to one embodiment, pressure regulator 20 is adjusted so that the flow of gas into the colon from tube 4, as measured by flow meter 14, is matched with the volume of gas drawn from the colon via the pressure regulator 20. According to another embodiment, no pressure regulator 20 is provided. Instead, pressure at the outflow of tube 3 and/or collection container 18 is at atmospheric pressure. A slight positive pressure is maintained in the colon due to the pressure drop caused by gas flow through the outflow tube 3 and collection container 18.

Connection between outflow tube 3 and collection container 18 is provided by a medically suitable connection, for example, by a Luer Lock connector or other medically suitable means of joining tubing or attaching tubing to a port. Preferably, all such connections are made in a sterile manner. For embodiments in which the proximal end of the outflow tube 3 extends to an effluent container 18, the length of the outflow tube is suitably adapted to a convenient length to reach the container or the attachment point.

The length of the distal portion of outflow tube 3 extending from stopper 2 is selected so that it can be comfortably disposed within the lumen of the rectum, sigmoid colon, and/or colon. According to one embodiment, the distal portion of the outflow tube 3 extending from stopper 2 has a length between about 2 and about 15 cm. According to a preferred embodiment, the length of the distal portion of outflow tube 3 is about 5 cm

According to one embodiment, the distal end of outflow tube 3 is open so that feces and gas delivered by gas delivery tube 4 into the colon are allowed to escape via tube 3. According to another embodiment instead of, or in addition to an opening at the distal end of outflow tube 3, outflow tube 3 has one or more openings along its length. The number, location, and dimensions of these openings are selected so that fecal matter that might be entrained in the flow of gas out from the colon does not clog the outflow tube 3. According to one embodiment, the diameter of the perforations is between about 1 mm and about 5 mm. According to one embodiment, perforations are provided at intervals of 1 to 5 cm along the distal portion of outflow tube 3.

As shown in the embodiment of FIG. 1, gas delivery tube 4 is disposed within the lumen of the colon and extends the anatomic length of the colon from the anus to the ileocecum valve. The distal tip may be placed anywhere along the length of the cecum, colon, or rectum. The length of tube 4 is selected so that, once the distal portion of tube 4 is positioned in the colon, a sufficient length of the tube 4 extending in the proximal direction from stopper 2 is provided to conveniently connect tube 4 with pressure converter 12, flow meter 14, and/or gas source 10. According to a preferred embodiment, from about 1 cm to up to about 100 cm of the proximal end of tube 4 extends from stopper 2.

In some embodiments, oxygen or a gas mixture is delivered to the colon via an opening at the distal end of delivery tube 4. According to this embodiment, tube 4 has no additional perforations. The distal end of tube 4 may be located near the ileocecum valve, as shown in FIG. 1. Alternatively, the distal end of tube 4 may be positioned at another location in the colon, including along the ascending, transverse, or descending colon to deliver gas to specific tissues or specific areas of those tissues. Radiographic tip 30 may be used to locate the distal end of tube 4 in a selected region of the colon. According to another embodiment, all of, or selected portions of, gas delivery tube 4 and/or outflow tube 3 are radio opaque or radio-translucent to allow the tube to be visualized radiographically, for example, using a fluoroscope to assure proper placement of tube 4 within the colon and placement of tube 3 in the rectum.

According to other embodiments, tube 4 has one or more orifices 6a, 6b, 6c along its length instead of, or in addition to, an opening at the distal end. According to one embodiment, orifices 6a, 6b, 6c are equal in diameter, and have a diameter range of 0.1-5.0 mm. According to one embodiment, orifices 6a, 6b, 6c are provide at intervals 1 or 2 cm apart along the entire length of the distal portion of tube 4 disposed within the lumen of the colon. According to another embodiment, orifices 6a, 6b, 6c consist of sets of openings, for example, two opposing openings on opposite sides of tube 4 at intervals of 5-10 cm along the tube. According to one embodiment, orifices 6a, 6b, 6c are positioned to correspond with particular sites within the lumen of the colon. Such sites can correspond to defined sections of tissue within the colon lengths For example, one set of orifices 6a may be positioned to be located in the rectum or descending colon, another set of openings 6b may be positioned to be located within the transverse colon, and a third set of openings 6c may be positioned to be located in the ascending colon. The disclosure is not limited to a particular arrangement of openings and any arrangement and number of openings is contemplated.

According to one embodiment, gas delivery tube 4, stopper 2, and outflow tube 3 are assembled as shown in FIG. 1, with gas delivery tubes 4 positioned within inlet port 2a of stopper 2 and outflow tube 3 positioned within outflow port 2b. According to this embodiment, gas delivery tube and outflow tube 3 are inserted through the anus of the patient so that gas delivery tube 4 is located along the lumen of the colon and outflow tube 3 is positioned within the rectum of the colon, as shown in FIG. 1. Stopper 2 is inserted into the anus and inflatable portion 22 is inflated to form a gas tight seal with the anus. According to one embodiment, gas delivery tube 4 is movable in the proximal and distal directions though the inlet port 2a to adjust the length of the distal portion of tube 4 extending into the colon. Sealing ring 8 is positioned against the proximal end of stopper 2 to assure a gas-tight seal between gas delivery tube 4 and stopper 2.

According to another embodiment, stopper 2 is provided separately from tubes 3, 4. To treat a patient, stopper 2 is first inserted into the anus. Gas delivery tube 4 is advanced through inlet port 2a of stopper 2 until it is positioned within the lumen of the colon at the desired location. Sealing ring 8 is positioned against the proximal end of stopper 2 to assure a gas-tight seal between gas delivery tube 4 and stopper 2. Outflow tube 3 is advanced through outlet port 2b of stopper until tube 3 is positioned within the rectum. Inflatable portion 22 is inflated to form a gas-tight seal with the anus.

The proximal end of gas delivery tube 4 is connected with gas source 10 via converter 12 and flow meter 14. Source 10 is capable of supplying the desired percentage of oxygen, typically from 21-100% oxygen, at a flow rate of from 0.1 to 5 L/min and at gauge pressure of about 2 mm Hg. The flow of gas is administered as described above for the desired amount of time, and repeated at intervals which can be determined by those of skill in the art in accordance with the disease or condition being treated. For example, in treating CDI, the treatment regimen is administered for a time in which to inhibit the vegetative growth of CDI and to provide time for commensal gut flora to establish growth and outcompete C. difficile. Because oxygen-containing gas is supplied continuously, physiologic absorption of oxygen by the tissues of the colon does not restore the colon to its normal physiological state until treatment is completed.

Once treatment is completed, inflatable portions 22 are deflated via tube 22a and stopper 2 and tube 4 are withdrawn from the patient. Stopper 2 and tubes 3,4 may be disposable or reusable (with proper cleaning and appropriate sterilization).

Whether or not disposable, stopper 2 and tubes 3,4 can be sterilized by methods known in the art consistent with the sterility fields and protocols for practice of colorectal procedures. Likewise, all or part of the insufflation device can be sterilized by methods known in the art and suitable for the materials used in the device and its components.

While some embodiments of the invention have been described by way of illustration, it will be apparent that the invention can be put into practice with many modifications, variations and adaptations, and with the use of numerous equivalents or alternative solutions that are within the scope of persons skilled in the art, without departing from the spirit of the invention or exceeding the scope of the claims.

All publications, patents, and patent applications are herein incorporated by reference in their entirety to the same extent as if each individual publication, patent or patent application was specifically and individually indicated to be incorporated by reference in its entirety.

### REFERENCES

Balsells E, Shi T, Leese C, et al. Global burden of Clostridium difficile infections: A systematic review and meta-analysis. J Glob Health. 2019; 9(1).
Bhangu A, Nepogodiev D, Gupta A, Torrance A, Singh P. Systematic review and meta-analysis of outcomes following emergency surgery for Clostridium difficile colitis. Br J Surg. 2012; 99:1501-1513.
Centers for Disease Control and Prevention. 2016 Annual Report for the Emerging Infections Program for Clostridium difficile Infection. 2016; 014(CDI):2015-2016. https://www.cdc.gov/hai/eip/Annual-CDI-Report-2016.html
Elixhauser A, Jhung M. Clostridium Difficile-Associated Disease in U.S. Hospitals, 1993-2005: Statistical Brief #50. Healthc Cost Util Proj Stat Briefs. 2006; 353(23):1-11.
Fairweather N, Smits W K, Mcbride S M, et al. Chemical and Stress Resistances of Clostridium difficile Spores and Vegetative Cells. Front Microbiol. 2016; 7(1698):1-13.
Fekety R, McFarland L V., Surawicz C M, Greenberg R N, Elmer G W, Mulligan M E. Recurrent Clostridium difficile diarrhea: Characteristics of and risk factors for patients enrolled in a prospective, randomized, double-blinded trial. Clin Infect Dis. 1997; 24(3):324-333.
Gould C V., Edwards J R, Cohen J, et al. Effect of nucleic acid amplification testing on population-based incidence rates of Clostridium difficile infection. Clin Infect Dis. 2013; 57(9):1304-1307.
Guh, Y. Mu, L. G. Winston, H. Johnston, D. Olson, M. M. Farley, L. E. Wilson, S. M. Holzbauer, E. C. Phipps, G. K. Dumyati, Z. G. Beldavs, M. A. Kainer, M. Karlsson DNG. Trends in U.S. Burden of Clostridioides difficile Infection and Outcomes. N Engl J Med. 2020; 382(14):1320-1330.
Leffler D A, Lamont J T. Clostridium difficile infection. N Engl J Med. 2015; 372(16):1539-1548.
McFarland L V., Surawicz C M, Rubin M, Fekety R, Elmer G W, Greenberg R N. Recurrent Clostridium Difficile Disease: Epidemiology and Clinical Characteristics. Infect Control Hosp Epidemiol. 1999; 20(01):43-50.
McDonald L C, Gerding D N, Johnson S, et al. Clinical Practice Guidelines for Clostridium difficile Infection in Adults and Children: 2017 Update by the Infectious Diseases Society of America (IDSA) and Society for Healthcare Epidemiology of America (SHEA). Clin Infect Dis. 2018; 66(7):e1-e48.
Peng Z, Jin D, Kim H B, et al. Update on antimicrobial resistance in Clostridium difficile: Resistance mechanisms and antimicrobial susceptibility testing. J Clin Microbiol. 2017; 55(7):1998-2008.
Sailhamer E A, Carson K, Chang Y, Zacharias N, Spaniolas K, Tabbara M, Alam H B, DeMoya M A, Velmahos G C. Fulminant Clostridium difficile colitis: patterns of care and predictors of mortality. Arch Surg. 2009 May; 144(5):433-9.
Sholeh M, Krutova M, Forouzesh M, et al. Antimicrobial resistance in Clostridioides (Clostridium) difficile derived from humans: a systematic review and meta-analysis. Antimicrob Resist Infect Control. 2020; 9(1):158.
Songer J G, Uzal F A. Clostridial enteric infections in pigs. J Vet Diagnostic Investig. 2005; 17(6):528-536.
Weese J S. Clostridium difficile in food innocent bystander or serious threat ? Eur Soc Clin Infect Dis. 2009; 16(1):3-10.
Weese J S, Toxopeus L, Arroyo L. Short Communications Clostridium difficile associated diarrhoea in horses within the community: predictors, clinical presentation and outcome. Equine Vet J. 2006; 38:185-188.

I claim:

1. A method of inhibiting growth of obligate anaerobic microorganisms in the colon of a subject which comprises
(a) administering a gas comprising from about 21% to about 100% oxygen via the anus into the lumen of the colon at a gentle/low flow rate to establish a oxygenated environment,
(b) providing for continuous gas release via the anus to equalize internal and external pressure, and
(c) maintaining said environment for a time sufficient to inhibit vegetative growth of said microorganisms.

2. The method of claim 1, which comprises treating a disease or disorder of the colon associated with obligate anaerobic microorganisms in a subject, wherein administering a gas comprises administering from about 21% to about 100% oxygen via the anus into the lumen of the colon at a flow rate to establish a oxygenated environment for a time sufficient to treat said disease or disorder.

3. The method of claim 2, wherein said disease is C. difficile infection (CDI).

4. The method of claim 2, wherein said disease or disorder is gut dysbiosis.

5. The method of claim 2, wherein the flow rate ranges from about 0.1 L/min to about 5 L/min and/or produces a partial pressure of oxygen of at least 5 mm Hg in the colon.

6. A method of treating a disease or disorder of the colon associated with obligate anaerobic microorganisms in a subject which comprises
(a) positioning an insufflation device comprising an anal stopper fitted with an outflow tube and an oxygen inlet tube, through the anus and into the colon of said subject by securing the stopper at the anal canal opening and advancing the inlet tube into the colon to a desired location between the iliocecal valve and the anus;
(b) gently insufflating a gas comprising from about 21% to 100% oxygen into the colon through the inlet tube at a flow rate sufficient to increase the oxygen partial pressure in the colon and to produce a oxygenated environment while providing continuous gas release via the outflow tube; and
(c) maintaining said pressure for sufficient time to inhibit vegetative growth of said anaerobic microorganisms.

7. The method of claim 6, wherein inhibiting vegetative growth of said microorganisms increases the number and/or diversity of beneficial microorganisms in the colon.

8. The method of claim 6, wherein said disease or disorder is C. difficile infection (CDI), including but not limited to primary CDI, recurrent CDI, fulminant CDI, and/or non-fulminant CDI in humans or mammals, or is gut dysbiosis.

9. The method of any claim 6, wherein the flow rate ranges from about 0.1 L/min to about 5 L/min and/or produces a partial pressure of oxygen of at least 5 mm Hg in the colon.

10. The method of claim 6, wherein said time is about 30 minutes, about 1 hour, about 2 hours, about 4 hours, about 8 hours, about 16 hours, about 1 day, about 2 days, about 3 days, about 4 days, about 5 days, about 6 days, to about a week, or about 10 days.

11. An insufflation device comprising:
an anal stopper adapted to form a gas-tight seal with an anus of an organism, wherein the anal stopper comprises an inlet port and an outlet port; and
a gas delivery tube comprising a distal portion adapted to be positioned within a lumen of a colon of the organism, wherein the gas delivery tube extends through the inlet port of the stopper and is adapted to be positioned along at least a portion of a colon of the organism.

12. The device of claim 11, comprising: a source of a gas, wherein the proximal end of the gas delivery tube is connected an output of the source and wherein the gas delivery tube comprises an inner lumen and one or more orifices extending from the inner lumen to a space surrounding the gas delivery tube, wherein, when the gas delivery tube extends along at least a portion of the colon of the organism, the gas flows from the source through the inner lumen and out from the inner lumen through the one or more orifices, wherein the gas delivery tube is slidingly engaged with the inlet port and movable in a proximal and a distal direction to adjust a length of a distal portion of the gas delivery tube along the colon.

13. The device of claim 12, wherein the stopper comprises an outflow tube extending through outlet port, wherein, when the stopper is disposed in the anus of the organism and the distal portion of the gas delivery tube is disposed in the colon, the gas flows through the one or more orifices into the colon of the organism and outward from the colon through the outflow tube.

14. The device of claim 13, wherein the outflow tube comprises a pressure regulator, wherein the pressure regulator maintains a pressure of the gas in the colon at a treatment pressure.

15. The device of claim 12, wherein the stopper comprises an inflatable portion adapted to receive an inflation fluid, wherein, when the stopper is positioned in the anus and when inflation fluid is force into the inflatable portion, a surface of the stopper is pressed against a wall of the anus to form a gas-tight seal between the surface of the stopper. and the anus.

16. The device of claim 12, wherein the one or more orifices comprise a plurality of sets of orifices, wherein a first set is positioned a first distance along the length of the tube from the stopper, wherein the first distance corresponds with a distance along the colon from the anus to a descending colon of the organism and, optionally, a second set of orifices is positioned a second distance along the length of the tube from the stopper, wherein the second distance corresponds with a distance along the colon from the anus to a transverse colon of the organism.

17. The device according to claim 12, wherein the gas delivery tube further comprises one or more radio-opaque markers.

18. The device according to claim 12, further comprising a safety valve connected with the gas delivery tube. wherein, when a gas pressure in the inner lumen of the gas delivery tube exceeds a safety pressure, the safely valve opens to vent the inner lumen.

19. The device of claim 12, wherein the gas comprises between about 21% oxygen and about 100% oxygen.

20. The device of claim 12, wherein the gas comprises an admixture of oxygen and one or more of air, nitrogen, helium, hydrogen, and carbon dioxide.

21. A device for treating a disease or disorder of the colon associated with obligate anaerobic microorganisms comprising:
a source of a gas at a first pressure;
a pressure converter connected with the source, wherein the pressure converter receives the gas at the first pressure and outputs the gas at a second pressure;
a gas delivery tube, wherein a proximal end of the gas delivery tube is connected an output of the pressure converter and wherein the gas delivery tube comprises an inner lumen and one or more orifices extending from the inner lumen to a space surrounding the gas delivery tube; and
a stopper adapted to engage with and form a gas-tight seal with an anus of the organism, wherein the stopper comprises an inlet port and an outlet port,
wherein the gas delivery tube extends through the inlet port and is adapted to be positioned along at least a portion of the colon of the organism, wherein the gas flows from the source, through the converter, through the inner lumen and out from the inner lumen through the one or more orifices, wherein the gas delivery tube is slidingly engaged with the inlet port and movable in a proximal and a distal direction to adjust a length of a distal portion of the gas delivery tube extending from the stopper.
